SEQUENCE LISTING (21 sequences)

SEQ ID NO: 1 - DNA, 1106 bp, Paspalum notatum
gaaaaaccgcaccgcgacacactccgacgagacggcattcgaataagcagcagcagcagaaaataagcaagaagtaatgggatccctcgccaagatcgtggccgtggcggccgtgctggcggcgctcgtcgccggcgggtcctgcggcccccccaaggtgccgcccggccccaacatcaccaccaactacaacggcaagtggctgccggccaaggccacctggtacggccagcccaacggcgccggacccgacgacaacggcggtgcgtgcgggatcaagaacgtgaacctgccgccctacaacggcttcacggcctgcggcaaccctcccatcttcaaggatggcaagggatgtggctcatgctacgagatcagatgcaataagccagagtgctccggccagccggtgacggtgttcatcaccgacatgaactacgagcccattgccccctaccacttcgacctcagcggcaaggccttcggcgccatggccaagcccggcctcaacgacaagctccgccactacggcatcttcgacctggagttcaggagggtccggtgcaagtaccagggcgggcagaagatcgtgttccacgtggagaagggctccaaccccaactacctagcgatgctggtcaagttcgtggcggacgacggcgacatcgtgctcatggagctcaaggagaagagctcggactggaagccgatgaagctctcctggggcgccatctggaggatggacacgcccaaggcgctagtgccccccttctccatccgcctcaccagcgagtccggcaagaaggtcatcgcccaggacgtcattcccgtgaactggaagcccgacacggtctacaattccaacgtccagttctagatcgacttgtttctttaccatgcatcatgcgcatgcacttgatccgtctttttttttccctatgaggcaatggacagcatgcatgcaccattaaccatatatatgttgagaattttgaattattgtaatatgtaatccggccgggcacacattttttgctcgatcgagtcaatcaatgccaaaacagtggtttttaaaaaaaaaaaaaaaaaaaaaaaaaacactgtcatgc

SEQ ID NO: 2 - protein, 265 aa, Paspalum notatum
MGSLAKIVAVAAVLAALVAGGSCGPPKVPPGPNITTNYNGKWLPAKATWYGQPNGAGPDDNGGACGIKNVNLPPYNGFTACGNPPIFKDGKGCGSCYEIRCNKPECSGQPVTVFITDMNYEPIAPYHFDLSGKAFGAMAKPGLNDKLRHYGIFDLEFRRVRCKYQGGQKIVFHVEKGSNPNYLAMLVKFVADDGDIVLMELKEKSSDWKPMKLSWGAIWRMDTPKALVPPFSIRLTSESGKKVIAQDVIPVNWKPDTVYNSNVQF

SEQ ID NO: 3 - DNA, 726 bp, Paspalum notatum
cctcgccaagatcgtggccgtggcggccgtgctggcggcgctcgtcgccggcgggtcctgcggcccccccaaggtgccgcccggccccaacatcaccaccaactacaacggcaagtggctgccggccaaggccacctggtacggccagcccaacggcgccggacccgacgacaacggcggtgcgtgcgggatcaagaacgtgaacctgccgccctacaacggcttcacggcctgcggcaaccctcccatcttcaaggatggcaagggatgtggctcatgctacgagatcagatgcaataagccagagtgctccggccagccggtgacggtgttcatcaccgacatgaactacgagcccattgccccctaccacttcgacctcagcggcaaggccttcggcgccatggccaagcccggcctcaacgacaagctccgccactacggcatcttcgacctggagttcaggagggtccggtgcaagtaccagggcgggcagaagatcgtgttccacgtggagaagggctccaaccccaactacctagcgatgctggtcaagttcgtggcggacgacggcgacatcgtgctcatggagctcaaggagaagagctcggactggaagccgatgaagctctcctggggcgccatctggaggatggacacgcccaaggcgctagtgccccccttctccatccgcctcaccagcgagtccggcaagaaggtcatcgc

SEQ ID NO: 4 - protein, 242 aa, Paspalum notatum
GPPKVPPGPNITTNYNGKWLPAKATWYGQPNGAGPDDNGGACGIKNVNLPPYNGFTACGNPPIFKDGKGCGSCYEIRCNKPECSGQPVTVFITDMNYEPIAPYHFDLSGKAFGAMAKPGLNDKLRHYGIFDLEFRRVRCKYQGGQKIVFHVEKGSNPNYLAMLVKFVADDGDIVLMELKEKSSDWKPMKLSWGAIWRMDTPKALVPPFSIRLTSESGKKVIAQDVIPVNWKPDTVYNSNVQF

SEQ ID NO: 5 - DNA, 35 bp, Artificial Sequence
  Oligonucleotide
  misc_feature (18)..(18): n is a or g
  misc_feature (27)..(27): n is a or g
gggcccccgaaggtggcncccggcaanaacatcac

SEQ ID NO: 6 - DNA, 24 bp, Artificial Sequence
  Oligonucleotide
  misc_feature (18)..(18): n is a, c, g, or t
gggcccccgaaggtggcncccggc

SEQ ID NO: 7 - DNA, 29 bp, Artificial Sequence
  Oligonucleotide
ggtgcctccctcggtagtgaggcggacgg

SEQ ID NO: 8 - DNA, 26 bp, Artificial Sequence
  Oligonucleotide
cccttcttggccatggcgccgaacgc

SEQ ID NO: 9 - DNA, 29 bp, Artificial Sequence
  Oligonucleotide
cggatcaagtgcatgcgcatgatgcatgg

SEQ ID NO: 10 - DNA, 34 bp, Artificial Sequence
  Oligonucleotide
gcttgcaagcttctagaactggacgttggaattg

SEQ ID NO: 11 - protein, 243 aa, Artificial sequence
  Consensus sequence
MSSSVLLVVALAVFGAGIPKVPPGPNITATYGDKWLDAKSTWYGKPTGAGPKDNGGACGYKDVDKAPFGMTGCGNTPIFKDGRGCGSCFEIKCKPECSGEPVTVHITDDNEEPIAPYHFDLSGAFGAMAKKGEEKLRAGELELQFRRVKCKYPGTKTFHVEKGSNPNYLALLVKYVGDGDVVAVDIKEKGKDKWILKESWGAIWRDTPDKLTGPFTVRYTTEGGTKEAEDVIPEGWKADTYSK

SEQ ID NO: 12 - protein, 263 aa, Lolium pratense
MASSSSVLLVVALFAVFLGSAHGIAKVPPGPNITAEYGDKWLDAKSTWYGKPTGAGPKDNGGACGYKNVDKAPFNGMTGCGNTPIFKDGRGCGSCFEIKCTKPESCSGEAVTVTITDDNEEPIAPYHFDLSGHAFGSMAKKGEEQNVRSAGELELQFRRVKCKYPDDTKPTFHVEKASNPNYLAILVKYVDGDGDVVAVDIKEKGKDKWIELKESWGAVWRIDTPDKLTGPFTVRYTTEGGTKSEFEDVIPEGWKADTSYSAK

SEQ ID NO: 13 - protein, 269 aa, Phalaris arundinacea
MMKMVCSSSSSSLLVVAALLAVFVGSAQGIAKVPPGPNITAEYGDKWLDAKSTWYGKPTGAGPKDNGGACGYKDVDKAPFNGMTGCGNTPIFKDGRGCGSCFELKCSKPESCSGEPITVHITDDNEEPIAPYHFDLSGHAFGSMAKKGEEENVRGAGELELQFRRVKCKYPDGTKPTFHVEKGSNPNYLALLVKYVDGDGDVVAVDIKEKGKDKWIELKESWGAIWRIDTPDKLTGPFTVRYTTEGGTKAEFEDVIPEGWKADTHDASK

SEQ ID NO: 14 - protein, 265 aa, Holcus lanatus
MASSSRSVLLLVAALFAVFLGSAHGIAKVPPGPNITATYGDEWLDAKSTWYGKPTGAGPKDNGGACGYKDVDKPPFSGMTGCGNTPIFKDGRGCGSCFEIKCTKPESCSGEPVTVHITDDNEEPIAPYHFDLSGHAFGSMAKKGEEQKLRSAGELELKFRRVKCKYPDGTKPTFHVEKGSNPNYLALLVKYIDGDGDVVAVDIKEKGKDKWIELKESWGAVWRVDTPDKLTGPFTVRYTTEGGTKGEAEDVIPEGWKADTAYEAK

SEQ ID NO: 15 - protein, 265 aa, Dactylis glomerata; identical to SEQ ID NO: 14

SEQ ID NO: 16 - protein, 263 aa, Phleum pratense
MASSSSVLLVVVLFAVFLGSAYGIPKVPPGPNITATYGDKWLDAKSTWYGKPTGAGPKDNGGACGYKDVDKPPFSGMTGCGNTPIFKSGRGCGSCFEIKCTKPEACSGEPVVVHITDDNEEPIAPYHFDLSGHAFGAMAKKGDEQKLRSAGELELQFRRVKCKYPEGTKVTFHVEKGSNPNYLALLVKYVNGDGDVVAVDIKEKGKDKWIELKESWGAIWRIDTPDKLTGPFTVRYTTEGGTKTEAEDVIPEGWKADTSYESK

SEQ ID NO: 17 - protein, 263 aa, Poa pratensis
MASSSSVLLVVALFAVFLGTAHGIAKVPPGPNITATYGDKWLDAKSTWYGKPTGAGPKDNGGACGYKDVDKAPFSGMTGCGNTPIFKSGRGCGSCFEIKCTKPESCSGEPVLVHITDDNEEPIAAYHFDLSGKAFGAMAKKGEEQKLRSAGELELKFRRVKCEYPEGTKVTFHVEKGSNPNYLALLVKYVTGDGDVVAVDIKEKGKDKWIELKESWGSIWRVDTPDKLTGPFTVRYTTEGGTKGEAEDVIPEGWKADTAYASK

SEQ ID NO: 18 - protein, 261 aa, Sorghum halepense
MSWSMQVALVVALAFLVGGAWCGPPKVAPGKNITATYGSDWLERKATWYGKPTGAGPDDNGGACGYKDVNKAPFNSMGACGNLPIFKDGLGCGSCFEIKCDKPAECSGEAVVVHITDMNYEQIAAYHFDLAGTAFGAMAKKGEEEKLRKAGIIDMKFRRVKCKYGEKVTFHVEKGSNPNYLALLVKYVDGDGDVVGVDIKEKGGDAYQPLKHSWGAIWRKDSDKPIKFPVTVQITTEGGTKTAYEDVIPEGWKADTTYTAK

SEQ ID NO: 19 - protein, 267 aa, Oryza sativa
MASSSLLLACVVVAAMVSAVSCGPPKVPPGPNITTSYGDKWLEAKATWYGAPKGAGPKDNGGACGYKDVDKAPFLGMNSCGNDPIFKDGKGCGSCFEIKCSKPEACSDKPALIHVTDMNDEPIAAYHFDLSGLAFGAMAKDGKDEELRKAGIIDTQFRRVKCKYPADTKITFHIEKASNPNYLALLVKYVAGDGDVVEVEIKEKGSEEWKALKESWGAIWRIDTPKPLKGPFSVRVTTEGGEKIIAEDAIPDGWKADSVYKSNVQAK

SEQ ID NO: 20 - protein, 262 aa, Cynodon dactylon
MLAAVAVVLASMVGGAWCAMGDKPGPNITATYGDKWLDAKATFYGSDPRGAAPDDHGGACGYKDVDKAPFDGMTGCGNEPIFKDGLGCGSCYEIKCKEPAECSGEPVLIKITDKNYEHIAAYHFDLSGKAFGAMAKKGEEDKLRKAGELMLQFRRVKCEYPSDTKITFHVEKGSSPNYLALLVKYAAGDGNIVGVDIKPKGSDVFLPMKLSWGAIWRMDPPKPLKGPFTIRLTSESGGHVEQEDVIPEDWKPDTVYKSKIQF

SEQ ID NO: 21 - protein, 269 aa, Zea mays
MGSLANNIMVVGAVLAALVVGGSCGPPKVPPGPNITTNYNGKWLTARATWYGQPNGAGAPDNGGACGIKNVNLPPYSGMTACGNVPIFKDGKGCGSCYEVRCKEKPECSGNPVTVFITDMNYEPIAPYHFDLSGKAFGSLAKPGLNDKLRHCGIMDVEFRRVRCKYPAGQKIVFHIEKGCNPNYVAVLVKFVADDGDIVLMEIQDKLSAEWKPMKLSWGAIWRMDTAKALKGPFSIRLTSESGKKVIAKDIIPANWRPDAVYTSNVQFY

(19) United States
(12) Patent Application Publication
(10) Pub. No.: US 2017/0276677 A1
(43) Pub. Date: Sep. 28, 2017

(54) DETECTION AND MEASUREMENT OF GRASS POLLEN PROTEINS

(71) Applicant: Queensland University of Technology, Brisbane (AU)

(72) Inventor: Janet Davies, Wooloongabba (AU)

(21) Appl. No.: 15/320,283

(22) PCT Filed: Jun. 22, 2015
(86) PCT No.: PCT/AU2015/050348

§ 371 Date: Dec. 20, 2016

Related U.S. Application Data
(60) Provisional application No. 62/015,324, filed on Jun. 20, 2014.

(51) Int. Cl. G01N 33/569; C07K 14/415; G01N 33/68; C07K 16/16; A61K 39/36; G01N 33/577

(52) U.S. Cl. CPC G01N 33/56961; A61K 39/36; G01N 33/577; G01N 33/68; C07K 16/16; C07K 14/415; G01N 2800/24

(57) ABSTRACT

Detection of grass pollen proteins such as Pas n 1, Sor h 1 and/or Cyn d 1 for the purpose of batch standardization of compositions comprising these grass pollen proteins, such as used for desensitization to these allergenic proteins. The methods may also be suitable for detecting or measuring levels of grass pollen proteins such as Pas n 1, Sor h 1 and/or Cyn d 1 in environmental samples such as air or water. Also provided is an antibody or antibody fragment engineered to bind Pas n 1 and one or a plurality of other grass pollen proteins such as Sor h 1 and/or Cyn d 1.

### TECHNICAL FIELD

The present invention relates generally to detection and/or measurement of grass pollen proteins. More particularly, the present invention is directed to detection and/or measurement of grass pollen allergen proteins Pas n 1, Cyn d 1 and/or Sor h 1 and derivatives, variants and fragments thereof for environmental monitoring and/or batch standardization of pharmaceutical or diagnostic compositions.

### BACKGROUND

Bibliographic details of the publications referred to by author in this specification are collected alphabetically at the end of the description.

The frequency of atopy and the incidence of allergic diseases, such as seasonal rhinitis, have increased in recent decades, hereby making the characterisation of causative allergens a priority (Robinson et al. Linnenborg et al., 2001). Grass pollen, in particular, are linked to the onset of allergy in a very significant proportion of the population.

Bahia grass, Paspalum notatum, is endemic to sub-tropical parts of the Americas near the equator and has been introduced elsewhere, including Australia, as lawn, feed crop or under-planting ground cover in orchards (Firth). Bahia grass is a perennial warm weather bunch grass with a deep root system that grows well on all soils in wet areas. Production of pollen is moderate to heavy. Bahia grass pollen is capable of provoking an immediate hypersensitivity type I reaction in patients who are allergic to either this grass source or to pollen of Bermuda grass or other members of the subfamily Pooideae. Previous studies also indicate that patients allergic to the pollen of some trees also show strong reactivity to Bahia grass. Whilst the major temperate grass species including Timothy and Ryegrass pollinate during spring and early summer, triggering seasonal allergic rhinitis and asthma in sensitized individuals, the pollination period of Bahia grass extends from spring through to autumn, thereby often triggering allergic symptoms late in the grass pollen season. Accordingly, Bahia grass is recognised as an important allergen source for triggering allergic rhinitis and seasonal asthma (Davies et al., 2005; Weber et al. 2006).

To this end, the Group I allergens of grass pollen are important because they are clinically significant allergens which play a vital biological role in fertilization of the grass. They are major allergens which react at a frequency greater than 90% with serum IgE of allergic individuals. The Group I allergens generally comprise up to 4% of the total pollen protein, making them a major component of the pollen (Yennawar et al., 2006). However, they are critical not only because of their abundance but because of their function.

In terms of Bahia-related allergy, it has been reported that immunotherapy with a mixture of Timothy and Bermuda grass pollen extract could diminish wheals induced by allergen skin prick testing in response to 10 grasses including Bahia grass. It would suggest that inclusion of Bahia grass pollen may not be required for an effective grass pollen immunotherapy extract for seasonal allergy (Phillips et al., 1989). However, whereas Bahia grass is allergenic in nasal challenges, it is not cross reactive with Timothy grass pollen by nasal challenge, indicating that immunotherapy vaccines lacking Bahia grass may not effectively treat Bahia grass pollen allergy (Phillips et al., 1989).

Only limited IgE cross-reactivity between ryegrass and Bahia grass has been found, consistent with the distinct phylogeny of these grasses (Davies et al., 2005). Bahia grass is phylogenetically distinct from ryegrass and Timothy grass. While these two and other well characterised allergenic grass species belong to the Pooideae subfamily of temperate grasses, Bahia grass resides in the Pancoideae sub-family.

In view of the importance of Bahia grass as an allergenic grass species, improved methods of detection and/or measurement of Bahia grass pollen allergens are required for applications including environmental monitoring of such allergens and batch standardization of pharmaceutical and diagnostic compositions including such allergens.

### SUMMARY

The invention is broadly directed to detection of one or more grass pollen proteins, or more particularly measurement and/or determination of an amount or level of the one or more grass pollen proteins. In some broad embodiments, the one or more grass pollen proteins are in an environmental sample such as air or water. In some other broad embodiments, the one or more grass pollen proteins are in a pharmaceutical composition such as for desensitizing a subject to the one or more grass pollen proteins. A particular embodiment relates to an antibody or antibody fragment that binds the grass pollen protein Pas n 1 and one or a plurality of other grass pollen proteins.

In a first aspect, the invention provides a method for detecting a Pas n 1 protein and/or measuring the level of the Pas n 1 protein in a sample, wherein the Pas n 1 protein comprises an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, said method comprising the step of contacting the sample with an antibody or antibody fragment that binds the Pas n 1 protein, fragment or derivative for a time and under conditions sufficient to allow the formation of a detectable antibody- or antibody fragment-protein complex.

Suitably, the sample comprises one or a plurality of other grass pollen-derived proteins in addition to said protein. In certain embodiments, the one or plurality of other grass pollen-derived proteins are selected from the group consisting of a Sor h 1 protein, a Cyn d 1 protein, a Lol p 1 protein, a Lol p 5 protein, a Lol p 11 protein, a Phl p 1 protein, a Dac g 1 protein, an Ant o 1 protein, a Pha a 1 protein, a Poa p 1 protein and a Pas n 13 protein. In a particular embodiment, the one or plurality of other grass pollen-derived proteins include a Sor h 1 protein and/or a Cyn d 1 protein.

In a second aspect, the invention provides a method for detecting a Cyn d 1 protein and/or a Sor h 1 protein and/or measuring the level of the Cyn d 1 protein and/or the Sor h 1 protein in a sample, said method comprising the step of contacting the sample with an antibody or antibody fragment that binds and/or is raised against a Pas n 1 protein comprising an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, for a time and under conditions sufficient to allow the formation of a detectable antibody- or antibody fragment-protein complex.

Suitably, the sample comprises one or a plurality of other grass pollen-derived proteins in addition to said Cyn d 1 protein and/or said Sor h 1 protein. In certain embodiments, the one or plurality of other grass pollen-derived proteins are selected from the group consisting of a Lol p 1 protein, a Lol p 5 protein, a Lol p 11 protein, a Phl p 1 protein, a Dac g 1 protein, an Ant o 1 protein, a Pha a 1 protein, a Poa p 1 protein, a Pas n 1 protein and a Pas n 13 protein.

Suitably, the method of the aforementioned aspects is for determining a relative and/or absolute amount of each of the proteins in the sample.

In certain embodiments, the antibody is capable of binding both the Pas n 1 protein and the one or a plurality of the other grass pollen-derived proteins. In one embodiment, the antibody is capable of binding the Pas n 1 protein and: (i) a Sor h 1 protein; (ii) a Cyn d 1 protein; or (iii) a Sor h 1 protein and a Cyn d 1 protein.

In one embodiment, the sample is an environmental sample, preferably air or water. In an alternative embodiment, the sample is, or is derived from, a pharmaceutical composition for immunotherapy. In another embodiment, the sample is, or is derived from, a diagnostic composition. With respect to these embodiments, the method is suitably performed to batch standardize the pharmaceutical composition or the diagnostic composition.

In a third aspect, the invention provides an antibody or antibody fragment that binds and/or is raised against a Pas n 1 protein comprising an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, wherein the antibody or antibody fragment also binds one or a plurality of other grass pollen proteins.

In a fourth aspect, the invention provides a method of producing an antibody or antibody fragment including the step of producing and/or selecting an antibody or antibody fragment that binds and/or is raised against a Pas n 1 protein that comprises an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, whereby the antibody or antibody fragment also binds one or a plurality of other grass pollen proteins.

In a fifth aspect is provided an antibody or antibody fragment produced by the method of the fourth aspect.

In an embodiment, the antibody or antibody fragment is a recombinant antibody or antibody fragment. In a particular embodiment, the recombinant antibody or antibody fragment comprises an amino acid sequence that is modified or engineered to facilitate binding to said one or more other grass pollen proteins.

In one embodiment, the one or a plurality of other grass pollen proteins includes a Sor h 1 protein and a Cyn d 1 protein.

Suitably, the antibody or antibody fragment of the third and fifth aspects is for use in the method of the first and second aspects.

In a sixth aspect, the invention provides a composition or kit comprising the antibody or antibody fragment of the third and fifth aspects.

Suitably, the composition or kit further comprises one or more reagents that facilitate detection and/or measurement of a complex comprising the antibody or antibody fragment and the protein(s).

### BRIEF DESCRIPTION OF THE FIGURES

FIG. 1 is a multiple sequence alignment of known Group I grass pollen allergens. Clustal W alignment of the isoform closest to Pas n 1 from each grass species identified by BLAST search of the SwissProt and SpTrEMBL databases. Sor h 1 sequence of Johnson grass obtained from Avjioglu (U.S. Pat. No. 5736149).

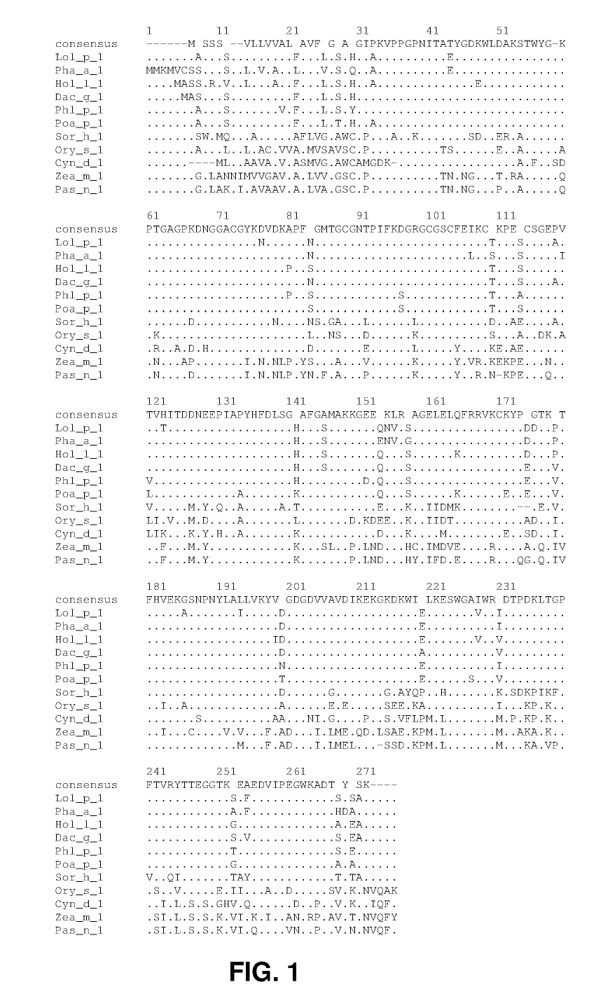

FIG. 2 demonstrates reactivity of monoclonal antibody producing hybridoma clones raised in mice by immunisation with subtropical grass pollen allergen Pas n 1. The culture supernatant of hybridoma clones STGP-B2, STGP-C2 and STGP-F2 were tested for IgG binding by enzyme linked immunosorbent assay with equal amounts of purified Pas n 1 of subtropical Bahia grass pollen, Sor h 1 of subtropical Johnson grass pollen, Cyn d 1 of subtropical Bermuda grass pollen, Lol 1 of the temperate Ryegrass pollen and Phl p 1 of the temperate Timothy grass pollen. Immunoreactivity absorbance values of hybridoma culture supernatants are shown minus background immunoreactivity values of wells coated with antigen and tested with media only negative controls (mean 1332, standard deviation 302 units).

### DETAILED DESCRIPTION

The invention is broadly directed to the detection and/or measurement of one or more grass pollen allergen proteins in an environmental sample or a sample that is, or is derived from, a pharmaceutical or diagnostic composition. In one broad embodiment, the environmental sample is air or water. In another broad embodiment the method is for batch standardization of a pharmacuetical composition or diagnostic composition that comprises the one or more grass pollen allergen proteins. The pharmaceutical composition may be for desensitizing a subject to the allergenic effect of the grass pollen protein(s). Typically, the one or more grass pollen allergen proteins are detected by an antibody or antibody fragment that binds each protein. In a particular embodiment, the antibody or antibody fragment binds and/or is raised against a Pas n 1 protein that is capable of binding one or more other grass pollen proteins such as Cyn d 1 and/or Sor h1. In particular embodiments, the antibody or antibody fragment is a recombinant antibody or antibody fragment that has been modified or engineered to facilitate binding the one or more other grass pollen proteins.

Throughout this specification and the claims that follow, unless the context requires otherwise, the word “comprise”, or variations such as “comprises” or “comprising” will be understood to imply the inclusion of a stated element or integer or group of elements or integers, but not the exclusion of any other element or integer or group of elements or integers.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The subject specification contains amino acid and nucleotide sequence information prepared using the programme PatentIn Version 3.1, presented herein after the bibliography. Each amino acid and nucleotide sequence is identified in the sequence listing by the numeric indicator <210> followed by the sequence identifier (eg. <210>1, <210>2, etc). The length, type of sequence (amino acid, DNA, etc.) and source organism for each sequence is indicated by information provided in the numeric indicator fields <211>m <212> and <213>, respectively. Amino acid and nucleotide sequences referred to in the specification are identified by the indicator SEQ ID NO: followed by the sequence identifier (eg. SEQ ID NO:1, SEQ ID NO: 2, etc). The sequence identifier referred to in the specification correlates to the information provided in numeric indicator field <400> in the sequence listing, which is followed by the sequence identifier (eg. <400>1, <400>2, etc). That is SEQ ID NO: 1 as detailed in the specification correlates to the sequence indicated as <400>1 in the sequence listing.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | The | T |
| Tryptophan | Trp | W |
| Tyrosine | Tyr | Y |
| Valine | Val | V |
| Any residue | Xaa | X |

In one aspect, the invention resides in a method for detecting a Pas n 1 protein and/or measuring the level of the Pas n 1 protein in a sample, said method comprising the step of contacting the sample with an antibody or antibody fragment that binds the Pas n 1 protein, fragment or derivative for a time and under conditions sufficient to allow the formation of a detectable antibody- or antibody fragment-protein complex.

Reference to “Pas n 1” should be understood as including reference to all forms of Pas n 1, or derivatives, variants or fragments thereof. Without limiting the present invention to any one theory or mode of action natural grass pollen allergens are well known to exhibit a wide variety of isoforms which may be expressed at different stages of maturation. Accordingly reference to “Pas n 1” should be understood to encompass all protein isoforms of Pas n 1 or derivatives, variants or fragments which may arise from alternative splicing of Pas n 1 mRNA. Reference to “Pas n 1” also includes reference to polymorphic variants of Pas n 1.

Without limiting the present invention to any one theory or mode of action, Pas n 1 is a major Group I pollen allergen from Bahia grass which is capable of triggering aberrant, unwanted or otherwise inappropriate immune response in subjects hypersensitive to grass pollen allergens, leading to conditions such as but not limited to allergic rhinitis or seasonal asthma. It can also induce hypersensitivity in individuals exhibiting a predisposition in this regard. Accordingly, it may be desirable to use Pas n 1 protein in a range of diagnostic and prophylactic or therapeutic treatment protocols for use with respect to Pas n 1 related conditions. Additionally, assessment of the presence or levels of a Pas n 1 protein in the environment may be of value to subjects with a Pas n 1 related condition.

Preferably, said Pas n 1 protein comprises, consists or consists essentially of the sequence set forth in SEQ ID NO: 2 or 4 or is a fragment, variant or derivative of said sequence.

By “consisting essentially of” means that the Pas n 1 protein comprises the amino acid sequence of SEQ ID NO: 2 or SEQ ID NO: 4 together with 1, 2, 3, 4 or 5 additional amino acids at the N- and/or C-terminus.

For the purposes of the present invention, SEQ ID NO:1 is, or corresponds to a nucleotide sequence of a Pas n 1 cDNA, including the nucleotide sequence encoding the signal peptide. SEQ ID NO:3 is, or corresponds to a nucleotide sequence encoding the Pas n 1 cDNA open reading frame of the mature protein, i.e. the protein without the signal sequence. SEQ ID NO:2 is, or corresponds to an amino acid sequence of a Pas n 1 protein which includes the signal sequence while SEQ ID NO:4 is, or corresponds to an amino acid sequence of a Pas n 1 protein which does not include the signal sequence.

By “protein” is meant an amino acid polymer. The amino acids may be natural or non-natural amino acids, D- or L-amino acids, as are well understood in the art.

A “peptide” is a protein having no more than sixty (60) amino acids.

A “polypeptide” is a protein having more than sixty (60) amino acids.

This aspect also includes fragments, variants and derivatives of said Pas n 1 protein.

For the purposes of the present invention, a protein “fragment” includes an amino acid sequence that constitutes less than 100%, but at least 20%, 30%, 40%, 50%, 60%, 70%, 80% or 90%, 92%, 94%, 96%, 98%, or 99% of said Pas n 1 protein (e.g., SEQ ID NO:2 and SEQ ID NO: 4).

In particular embodiments, a protein fragment may comprise, for example, at least 10, 15, 20, 25, 30 35, 40, 45, 50, 55, 60, 65, 70, 75, 80, 85, 90, 95, 100, 110, 120, 130, 140, 150, 160, 170, 180, 190, 200, 210, 220, 230, 240 and 250 contiguous amino acids of said Pas n 1 protein (e.g., SEQ ID NO:2 and SEQ ID NO: 4).

It will be appreciated that a peptide may be a protein fragment, for example comprising at least 6, 10, 12 preferably at least 15, 20, 25, 30, 35, 40, 45, and more preferably at least 50 contiguous amino acids.

Peptide fragments may be obtained through the application of standard recombinant nucleic acid techniques or synthesized using conventional liquid or solid phase synthesis techniques. For example, reference may be made to solution synthesis or solid phase synthesis as described, for example, in Chapter 18 of CURRENT PROTOCOLS IN PROTEIN SCIENCE, Coligan et al. Eds (John Wiley & Sons, 1995-2000). Alternatively, peptides can be produced by digestion of an isolated protein of the invention with proteases such as endoLys-C, endoArg-C, endoGlu-C and staphylococcus V8-protease. The digested fragments can be purified by, for example, high performance liquid chromatographic (HPLC) techniques as are well known in the art.

It will also be appreciated that larger peptides and isolated proteins comprising a plurality of the same or different Pas n 1 protein fragments are contemplated.

The invention also provides variants of a Pas n 1 protein.

As used herein, a protein “variant” shares a definable nucleotide or amino acid sequence relationship with an isolated protein disclosed herein. Preferably, Pas n 1 protein variants share at least 70% or 75%, preferably at least 80% or 85% or more preferably at least 90%, 91%, 92%, 93%, 94%, 95%, 96%, 97%, 98% or 99% sequence identity with a wildtype Pas n 1 amino acid sequence, such as SEQ ID NO: 2 or SEQ ID NO: 4.

Accordingly, in one embodiment, the Pas n 1 protein comprises an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA). Preferably, sequence identity is measured over the entire amino acid sequence of the Pas n 1 protein.

As used herein “variant” proteins disclosed herein have one or more amino acids deleted or substituted by different amino acids. It is well understood in the art that some amino acids may be substituted or deleted without changing the activity of the isolated protein (conservative substitutions).

The term “variant” includes peptidomimetics and orthologs of a Pas n 1 protein, such as that comprising an amino acid sequence set forth in SEQ ID NOS:2 or 4. By “peptidomimetic” is meant a molecule containing non-peptidic structural elements that are capable of mimicking or antagonising the biological action(s) of a natural parent peptide. Examples of peptidomimetics include peptidic compounds in which the peptide backbone is substituted with one or more benzodiazepine molecules (see, e.g., James et al., Science 260:1937-42, 1993) and “retro-inverso” peptides (see, e.g., U.S. Pat. No. 4,522,752). The term also refers to a moiety, other than a naturally occurring amino acid, that conformationally and functionally serves as a substitute for a particular amino acid in a protein without adversely interfering to a significant extent with the function of the protein. Examples of amino acid mimetics include D-amino acids. Proteins substituted with one or more D-amino acids may be made using well known peptide synthesis procedures. Additional substitutions include amino acid analogs having variant side chains with functional groups, such as, for example, b-cyanoalanine, canavanine, djenkolic acid, norleucine, 3-phosphoserine, homoserine, dihydroxyphenylalanine, 5-hydroxytryptophan, 1-methylhistidine, and 3-methylhistidine.

The term “variant” also includes Pas n 1 proteins or fragments thereof disclosed herein, produced from, or comprising amino acid sequences of, naturally occurring (e.g., allelic) variants, orthologs (e.g., from Paspalum spp., other than Paspalum notatum) and synthetic variants, such as produced in vitro using mutagenesis techniques.

Variant proteins or proteins can be produced by a variety of standard, mutagenic procedures known to one of skill in the art, such as those described herein.

Derivatives of the Pas n 1 protein are also provided.

As used herein, “derivative” proteins have been altered, for example by conjugation or complexing with other chemical moieties, by post-translational modification (e.g., phosphorylation, acetylation and the like), modification of glycosylation (e.g., adding, removing or altering glycosylation) and/or inclusion of additional amino acid sequences as would be understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g., polyhistidine) fusion partners, maltose binding protein (MBP), Protein A, glutathione S-transferase (GST), fluorescent protein sequences (e.g., GFP), epitope tags such as myc, FLAG and haemagglutinin tags.

Other derivatives contemplated by the invention include, but are not limited to, modification to side chains, incorporation of unnatural amino acids and/or their derivatives during peptide, or protein synthesis and the use of crosslinkers and other methods which impose conformational constraints on the isolated Pas n 1 proteins, fragments and variants of the invention.

As generally used herein, an antibody or antibody fragment is, or comprises, an immunoglubulin protein which may be monoclonal or polyclonal, native or recombinant. Preferably, the antibody or antibody fragment is a monoclonal antibody or a fragment thereof. Well-known protocols applicable to antibody production, purification and use may be found, for example in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with a Pas n 1 protein, fragment, variant, or derivative, such as that described herein. For example, the antibodies may be polyclonal antibodies. Such antibodies may be prepared for example by injecting an isolated Pas n 1 protein, fragment, variant or derivative hereinbefore described into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more isolated Pas n 1 proteins, fragments, variants or derivatives, such as those provided herein.

The invention also includes within its scope antibody fragments, such as Fc, Fab or F(ab)2 fragments of the polyclonal or monoclonal antibodies referred to above. Alternatively, the antibodies may comprise single chain Fv antibodies (scFvs) against the peptides of the invention. Such scFvs may be prepared, for example, in accordance with the methods described respectively in U.S. Pat. No 5,091,513, European Patent No 239,400 or the article by Winter & Milstein, 1991, Nature 349:293, which are incorporated herein by reference. The invention is also contemplated to include multivalent recombinant antibody fragments, so-called diabodies, triabodies and/or tetrabodies, comprising a plurality of scFvs, as well as dimerisation-activated demibodies (e.g., WO/2007/062466). By way of example, such antibodies may be prepared in accordance with the methods described in Holliger et al., 1993 Proc Natl Acad Sci USA 90:6444-6448; or in Kipriyanov, 2009 Methods Mol Biol 562:177-93 and herein incorporated by reference in their entirety.

In one embodiment, the antibody or antibody fragment binds to and/or is raised against a Pas n 1 protein comprising, consisting or consisting essentially of an amino acid sequence set forth in SEQ ID NO: 2 or SEQ ID NO: 4, or a fragment, variant or derivative thereof.

In particular embodiments, the antibody of the present invention specifically binds Pas n 1 or derivative, fragment or mutant thereof. By “specifically binds” is meant high avidity and/or high affinity binding of an antibody to a specific antigen. Antibody binding to its epitope on this specific antigen is stronger than binding of the same antibody to any other epitope, particularly those that may be present in molecules in association with, or in the same sample, as the specific antigen of interest. Antibodies that bind specifically to a protein of interest may be capable of binding other proteins at a weak, yet detectable, level. Such weak binding, or background binding, is readily discernible from the specific antibody binding to the protein of interest, e.g. by use of appropriate controls.

In alternative embodiments, the antibody or antibody fragment may bind Pas n 1 protein and one or a plurality of other grass pollen proteins, as will be described in more detail hereinafter.

Detecting grass pollen proteins such as Pas n 1, Sor h 1 and/or Cyn d 1 may be useful, for example, in terms qualitatively or quantitatively detect or measure protein levels. A variety of methods well known in the art can be used to determine anti-body-protein complex formation and/or levels either directly or indirectly. Such methods include immunochemical methods, such as western blotting, ELISA, immunoprecipitation, microbead assay and RIA, gel electrophoresis methods including one and two-dimensional gels, methods based on protein or peptide chromatographic separation or methods that use protein-fusion reporter constructs and colorimetric readouts.

A wide range of immunoassay techniques may be used such as those described in U.S. Pat. Nos. 4,016,043, 4,424,279 and 4,018,653. These methods may be employed for detecting grass pollen proteins such as Pas n 1, Sor h 1 and/or Cyn d 1. By way of example only, an antibody raised against Pas n 1 is immobilised onto a solid substrate to form a first complex and an environmental sample to be tested for the presence of Pas n 1, Sor h 1 and/or Cyn d 1 brought into contact with the bound molecule. After a suitable period of incubation, for a period of time sufficient to allow formation of an antibody-grass pollen protein secondary complex, a second Pas n 1 antibody labelled with a reporter molecule capable of producing a detectable signal is then added and incubated, allowing sufficient time for the formation of a tertiary complex of antibody-grass pollen protein—labelled antibody. Any unreacted material is washed away, and the presence of the tertiary complex is determined by observation of a signal produced by the reporter molecule. The results may either be qualitative, by simple observation of the visible signal or may be quantitated by comparison with a control sample containing known amounts of hapten. Variations of this assay include a simultaneous assay, in which both sample and labelled antibody are added simultaneously to the bound antibody, or a reverse assay in which the labelled antibody and sample to be tested are first combined, incubated and then added simultaneously to the bound antibody. These techniques are well known to those skilled in the art, and the possibility of minor variations will be readily apparent. This method may also be adapted to screen for the presence of antibody in a sample using the protein molecule, or fragments thereof, of the present invention.

By “reporter molecule”, as used in the present specification, is meant a molecule which, by its chemical nature, produces an analytically identifiable signal which allows the detection of antigen-bound antibody. Detection may be either qualitative or quantitative. The most commonly used reporter molecule in this type of assay are either enzymes, fluorophores or radionuclide containing molecules (i.e. radioisotopes). In the case of an enzyme immunoassay, an enzyme is conjugated to the second antibody, generally by means of glutaraldehyde or periodate. As will be readily recognised, however, a wide variety of different conjugation techniques exist which are readily available to one skilled in the art. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. It is also possible to employ fluorogenic substrates, which yield a fluorescent product.

Alternatively, fluorescent compounds, such as fluorescein, europium and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining complex is then exposed to the light of the appropriate wavelength, the fluorescence observed indicates the presence of the molecule of interest. Immunofluorescence and EIA techniques, including monitoring by time resolved fluorescence, are both very well established in the art. However, other reporter molecules, such as radioisotope, chemiluminescent or bioluminescent molecules, may also be employed. It will be readily apparent to the skilled technician how to vary the procedure to suit the required purpose.

In some embodiments, for detection and/or measurement of Pas n 1 and optionally one or more other grass pollen proteins such as Sor h 1 and/or Cyn d 1, the sample may be an environmental sample, inclusive of indoor and outdoor environmental samples. In this regard, detection of a Pas n 1 protein in environmental samples may assist monitoring the levels of grass pollens and their allergens such as the Pas n 1 protein in the environment. Environmental samples may include air, water, soil and food, although without limitation thereto. This may assist in providing advice or warning to allergy sufferers that preventative steps should be taken before entering environments that contain potentially high or dangerous levels of grass pollen proteins such as Pas n 1 that could cause adverse clinical outcomes such as an allergic response. This may also assist in clinical decisions on appropriate use of allergen-specific immunotherapy based on knowledge of exposure to grass pollen protein, such as Pas n 1, in the local environment. In a particular embodiment, the grass pollen protein Pas n 1 protein is detected in an assay using an antibody to Pas n 1 as disclosed herein. Such assays, as would be readily understood by those skilled in the art, may include immunoassays, such as western blot, multiplex bead array and ELISA. It should be understood, however, that this invention is not limited by reference to these specific methods of detection or immunoassay disclosed.

This particular embodiment of the invention may involve the acquisition of indoor samples, such as from homes, schools, commercial buildings and workplaces, and/or outdoor samples. For example, to detect and/or monitor pollen allergen levels in a household environment, a suitable sample may be collected dust. Preferably, the environmental sample is air or water.

Suitably, the level of sensitivity is such that it will detect allergens which are present in the environment in concentrations at least which are just high enough to be clinically significant in that they are likely to elicit an immune response in a sensitive subject.

In another embodiment, the sample is, or is derived from either a pharmaceutical composition for immunotherapy. Immunotherapy typically involves administration of an extract of the allergen source, such as a whole pollen extract, containing said allergen, such as a Pas n 1 protein or fragments, variants or derivatives thereof, to a patient as a means of desensitising or inducing immunological tolerance to said allergen. Such treatment may be based, for example, on the administration of specific concentrations of a given protein allergen, such as that present within an extract of an allergen source, in accordance with a specific regime in order to induce tolerance or reduction in the severity of hypersensitivity. Accordingly, the present method may be suitable for the production of a pharmaceutical composition for immunotherapy, such as a whole pollen extract, with a standardized content of an allergen protein (e.g., Pas n 1, Sor h 1, Cyn d 1).

In a further embodiment, the sample is, or is derived from a diagnostic composition. The diagnostic composition may be used in diagnostic applications including, but not limited to, measurement of the reactivity of a subject's cells to Pas n 1. This is of use, for example, with respect to the diagnosis and/or monitoring of conditions characterised by an aberrant, unwanted or otherwise inappropriate immune response, such as hypersensitivity or allergy, to Pas n 1. The proteins may be added into solution or bound to a solid support together with cells derived from peripheral blood or from tissue either unfractionated, fractionated or derived as a continuous cell line. Reactivity to the subject protein may then be measured by standard proliferation assays such as incorporation of H³-thymidine, measurement of expressed or secreted molecules such as surface markers, cytokines or other standard assays of cellular activity which are well known in the art. One may also use the diagnostic composition to screen for the presence of Pas n 1 specific antibodies (e.g., of the IgE and/or IgG classes and particularly those of the IgG4 subclass) in a biological sample, such as a blood sample, derived from a subject. Additionally, such diagnostic compositions may be used in in vivo provocation testing, such as skin testing (e.g., skin prick testing), nasal provocation testing, allergen aerosol chamber challenge, bronchial provocation testing or food challenge testing

It is well appreciated that validated assays are required for the quality control of diagnostic and pharmaceutical products. These are applied at various stages of the manufacturing process to confirm batch-to-batch reproducibility and for final product clearance and release. Indeed, specifications and target values and stability data are typically submitted to regulatory bodies as part of the registration process for such compositions. Amongst the most important requirement is the need for standardisation of the potency, efficacy or levels of the allergen protein (e.g. Pas n 1) in the composition to ensure batch-to-batch consistency (i.e. batch standardisation).

In one particular embodiment of the present method, the pharmaceutical composition or the diagnostic composition are batch standardised according to a level of a Pas n 1 protein.

It would be understood that the pharmaceutical and/or diagnostic compositions described herein may comprise, for example, one or a plurality of whole pollen extracts (e.g., from Bahia grass, Johnson grass and/or Bermuda grass) and/or one or a plurality of recombinant proteins (e.g., Pas n 1, Sor h 1 and/or Cyn d 1). Accordingly, the aforementioned disclosure is also applicable to detection and/or measurement of other grass pollen proteins such as Sor h 1 and/or Cyn d 1 alone or together with Pas n 1.

Once the level, potency or efficacy of the sample is determined, the composition may be processed in a way, such as purifying, concentrating or solubilising, to make it more suitable for the subsequent use in diagnosis or therapy. In a particular embodiment, the Pas n 1 protein is detected in an assay using an antibody to Pas n 1 as disclosed herein. Such assays, as would be readily understood by those skilled in the art, may include immunoassays, such as western blot and ELISA. It should be understood, however, that this invention is not limited by reference to these specific methods of detection or immunoassay disclosed.

Preferably, the antibodies of this aspect will be provided in molar excess to the levels of allergen protein that would be expected to be detected in a typical test sample.

In one embodiment, the sample comprises one or a plurality of other grass pollen-derived proteins or allergens in addition to said protein or allergen. Such grass pollen-derived proteins or allergens may include one or more of those described herein.

Suitably, the method of this aspect is for determining a relative and/or absolute amount of each of the proteins or allergens (e.g., Pas n 1 and/or the one or plurality of the grass pollen—derived proteins or allergens such as Cyn d 1 and/or Sor h 1) in the sample. Preferably, the levels of allergen protein detected in the test sample will be quantifiable.

In particular embodiments of the method of the present aspect, the sample further comprises one or a plurality of other grass pollen-derived proteins or allergens in addition to said Pas n 1 protein or allergen.

As previously described, detection of Pas n 1 in a sample may be be combined with detection of other grass pollen proteins to provide information about the presence or levels of a plurality of different grass pollen proteins in addition to Pas n 1. In this regard and without limiting the present invention in any way, subjects presenting with hypersensitivity to Bahia grass pollen, including the Pas n 1 protein or allergen, may also present with hypersensitivity to other grass pollens, such as but not limited to those of Johnson (Sorghum halepense), Timothy (Phleum pratense), Bermuda (Cynodon dactylon), Orchard grass (Dactylis glomerata), Sweet vernal grass (Anthoxanthum odoratum), Canary grass (Phalaris aquatica), Kentucky Blue grass (Poa pratensis) or Ryegrass (Lolium perenne).

As such, from a diagnostic stand point it may be important to assess the presence of hypersensitivity to other grass pollen-derived proteins or allergens, in addition to that of Pas n 1, in a subject. Furthermore, for immunotherapy it may be desirable to administer Pas n 1 in combination with one or more other grass pollen-derived proteins or allergens, including other Group I pollen allergens of the beta-expansin protein family, to a subject diagnosed with hypersensitivity and/or allergy to Pas n 1 and said other grass pollen-derived allergens. Accordingly, it may be of value to detect and/or measure the level of one or more other grass pollen-derived proteins or allergens, in addition to that of Pas n 1, in a pharmaceutical composition for immunotherapy and/or a diagnostic composition. Additionally, for environmental monitoring it may also be of value to detect and/or measure the level of one or more other grass pollen-derived proteins or allergens, in addition to that of Pas n 1, in an environmental sample.

As would be readily appreciated, the other grass pollen-derived proteins or allergens hereinbefore described may possess a number of isoforms which may be expressed at different stages of maturation. Indeed, the invention should be understood to encompass all protein isoforms of said other grass pollen-derived proteins or allergens, as are known in the art, or derivatives, variants or fragments thereof, including alternative splice forms and polymorphic variants thereof.

Suitably, the one or plurality of other grass pollen-derived proteins or allergens include one or a plurality of grass pollen-derived proteins or allergens from Bahia grass (Paspalum notatum), Johnson grass (Sorghum halepense), Bermuda grass (Cynodon dactylon), Ryegrass (Lolium perenne), Timothy grass (Phleum pratense), Orchard grass (Dactylis glomerata), Sweet vernal grass (Anthoxanthum odoratum), Canary grass (Phalaris aquatica) and Kentucky Blue grass (Poa pratensis).

Suitably, the other grass pollen-derived proteins or allergens from Johnson grass may be selected from a Sor h 1 protein, a Sor h 2 protein and a Sor h 3 protein. Preferably, the other grass pollen-derived protein/s or allergen/s from Johnson grass are selected from one or more of those isoforms provided in Davies (WO 2014/201499 A1) and/or Avjioglu (U.S. Pat. No. 5736149), which are incorporated by reference herein.

Suitably, the other grass pollen-derived protein/s or allergen/s from Bermuda grass may be selected from a Cyn d 1 protein, a Cyn d 2 protein, a Cyn d 4 protein, a Cyn d 6 protein, a Cyn d 7 protein, a Cyn d 11 protein, a Cyn d 12 protein, a Cyn d 13 protein, a Cyn d 15 protein, a Cyn d 22 protein, a Cyn d 23 protein and a Cyn d 24 protein. Preferably, the other grass pollen-derived protein or allergen from Bermuda grass is a Cyn d 1 protein. Even more preferably, the other grass pollen-derived protein/s or allergen/s from Bermuda grass is selected from one or more of those isoforms provided in O'Hehir et al. (US 2011/0217325 A1), which is incorporated by reference herein.

Suitably, the other grass pollen-derived protein/s or allergen/s from Ryegrass may be selected from a Lol p 1 protein, a Lol p 2 protein, a Lol p 3 protein, a Lol p 4 protein, a Lol p 5 protein, a Lol p 7 protein, a Lol p 10 protein, a Lol p 11 protein, a Lol p 12 protein and a Lol p 13 protein.

Preferably, the other grass pollen-derived protein or allergen from Ryegrass is a Lol p 1 protein, a Lol p 5 protein or a Lol p 11 protein.

Suitably, the other grass pollen-derived protein or allergen from Bahia grass is a Pas n 13 protein.

In one embodiment, the other grass pollen-derived protein or allergen from Timothy grass is a Phl p 1 protein.

In one embodiment, the other grass pollen-derived protein or allergen from Orchard grass is a Dac g 1 protein.

In one embodiment, the other grass pollen-derived protein or allergen from Sweet Vernal grass is an Ant o 1 protein.

In one embodiment, the other grass pollen-derived protein or allergen from Canary grass is a Pha a 1 protein.

In one embodiment, the other grass pollen-derived protein or allergen from Kentucky Blue grass is a Poa p 1 protein.

Preferably, the one or plurality of other grass pollen-derived proteins or allergens are from one or more subtropical grasses, such as Bahia grass (Paspalum notatum), Johnson grass (Sorghum halepense) and/or Bermuda grass (Cynodon dactylon). In one preferred embodiment, the one or plurality of other grass pollen-derived proteins or allergens are selected from the group consisting of Sor h 1 and Cyn d 1.

In a particular aspect the invention therefore provides an antibody or antibody fragment that binds and/or is raised against a Pas n 1 protein comprising an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, wherein the antibody or antibody fragment also binds one or a plurality of other grass pollen proteins.

A related aspect of the invention provides a method of producing an antibody or antibody fragment including the step of producing and/or selecting an antibody or antibody fragment that binds and/or is raised against a Pas n 1 protein that comprises an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, whereby the antibody or antibody fragment also binds one or a plurality of other grass pollen proteins.

In a related aspect, the invention provides an antibody or antibody fragment produced by the method of the previous aspect.

Suitably, the antibody or antibody fragment of these aspects is a recombinant antibody or antibody fragment. In one embodiment, the recombinant antibody or antibody fragment comprises an amino acid sequence that is modified or engineered to facilitate binding to said one or more other grass pollen proteins, such as those provided herein. Preferably, the amino acid sequence is modified or engineered to facilitate binding to a Sor h 1 protein and/or a Cyn d 1 protein. Such antibodies may be modified or engineered by any method known in the art, including those described herein.

In this regard, a modified or engineered recombinant antibody or antibody fragment may be obtained through the application of standard antibody engineering and selected techniques as are known in the art. For example, reference may be made to, for example, Chapter 2 of APPLICATIONS AND ENGINEERING OF MONOCLONAL ANTIBODIES, David J. King, (Taylor & Francis, 1998) or the more recent review ANTIBODY ENGINEERING AND MODIFICATION TECHNOLOGIES by David Filpula (Biomolecular Engineering, vol 24, 2007, pages 201-215).

Typically, an amino acid sequence of a variable (V) region of an immunoglobulin heavy and/or light chain (one or more of the heavy and/or light chain CDRs) may be modified by the substitution of one or a plurality of amino acids to thereby modify the antigen-binding properties of an antibody or antibody fragment. The modified antibody or antibody fragment may then be selected according to desired antigen-binding properties.

Preferably, the amino acid sequence of the antibody or antibody fragment has been modified or engineered to facilitate binding to a Sor h 1 protein and/or a Cyn d 1 protein.

In embodiments of these aspects, the antibody or antibody fragment is capable of binding both the Pas n 1 protein and one or a plurality of other grass pollen-derived proteins, such as those described herein. In this regard, the antibody may be capable of binding any combination of the Pas n 1 protein and one, two three or more other grass pollen-derived proteins, such as those hereinbefore described. In particular embodiments, the antibody is capable of binding a Pas n 1 protein (or a fragment, variant or derivative thereof) and either: (i) a Sor h 1 protein (or a fragment, variant or derivative thereof); (ii) a Cyn d 1 protein (or a fragment, variant or derivative thereof); or (iii) a Sor h 1 protein and a Cyn d 1 protein (or a fragment, variant or derivative thereof).

In this regard, the antibody or antibody fragment described herein may be a recombinant antibody or antibody fragment comprising an amino acid sequence that has been modified or engineered to facilitate binding to said Pas n 1 protein and/or said one or more other grass pollen-derived proteins, such as described herein. In this regard, the recombinant antibody or antibody fragment may be modified or engineered and then selected by virtue of an increased or enhanced affinity of binding of said antibody or antibody fragment to said Pas n 1 protein and/or said one or more other grass pollen-derived proteins. Such binding affinity may be determined by any method known in the art. It would be understood that prior to the recombinant antibody or antibody fragment being modified or engineered, said antibody or antibody fragment may demonstrate little or no binding or affinity for said one or more other grass pollen-derived proteins. Furthermore, it would be appreciated that such engineering or modification may also be performed so as to increase or enhance binding affinity of the antibody or antibody fragment to Pas n 1.

Another aspect of the invention provides a method for detecting a Cyn d 1 protein and/or a Sor h 1 protein and/or measuring the level of the Cyn d 1 protein and/or the Sor h 1 protein in a sample, said method comprising the step of contacting the sample with an antibody or antibody fragment that binds and/or is raised against a Pas n 1 protein comprising an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, for a time and under conditions sufficient to allow the formation of a detectable antibody- or antibody fragment-protein complex.

Suitably, the sample comprises one or a plurality of other grass pollen-derived proteins in addition to said Cyn d 1 protein and/or said Sor h 1 protein. In certain embodiments, the one or plurality of other grass pollen-derived proteins are selected from the group consisting of a Lol p 1 protein, a Lol p 5 protein, a Lol p 11 protein, a Pas n 1 protein and a Pas n 13 protein.

Suitably, the method of this aspect is for determining a relative and/or absolute amount of each of the proteins (e.g., Sor h 1, Cyn d 1 and/or the one or plurality of other grass pollen-derived proteins) in the sample.

In certain embodiments, the antibody is capable of binding both the Pas n 1 protein and the one or a plurality of the other grass pollen-derived proteins. In one embodiment, the antibody is capable of binding the Pas n 1 protein and: (i) a Sor h 1 protein; (ii) a Cyn d 1 protein; or (iii) a Sor h 1 protein and a Cyn d 1 protein.

In one embodiment, the sample is an environmental sample, including those hereinbefore described, such as air or water.

In an alternative embodiment, the sample is, or is derived from, a pharmaceutical composition for immunotherapy. In another embodiment, the sample is, or is derived from, a diagnostic composition. With respect to these embodiments, the method is suitably performed to batch standardize the pharmaceutical composition or the diagnostic composition.

A yet further aspect of the invention provides a composition or kit comprising the antibody or antibody fragment described herein.

In particular embodiments, the composition or kit further comprises one or more reagents that facilitate detection and/or measurement of a complex comprising the antibody or antibody fragment and the protein(s). In this regard, the one or more reagents may comprise one or more of reporter molecules, such as enzymes, fluorophores and radioisotopes, as are known in the art, including those described herein.

The present invention is further described by reference to the following non-limiting Figures and Examples.

### EXAMPLE 1

### Detection and Measurement of Pas n 1

### Methods

### Hybridoma and mAb Production

Hybridoma clones STGP-B2, STGP-C2 and STGP-F2 were produced by injecting a purified full length Pas n 1 (SEQ ID NOS: 2 and 4) as the primary immunogen followed by a booster injection again with purified full length Pas n 1 (SEQ ID NOS: 2 and 4) and a final booster injection with purified full length Sor h 1 (Campbell et al., 2015, Journal of Allergy and Clinical Immunology) into mice. Antibody-producing cells were then collected from the mice's spleens, and fused with a myeloma cell. Mouse monoclonal antibodies were then derived from hybridoma clones STGP-B2, STGP-C2 and STGP-F2. ELISA protocol for mAb clone screening

Microtitre plates (96 well) were coated with Pas n 1, Sor h 1, Cyn d 1, Lol p 1 or Phl p 1 antigen at 0.1 microgram per well with sodium hydrogen carbonate buffer and blocked with bovine serum albumin in phosphate buffered saline. Monoclonal antibody hybridoma culture supernatants were incubated undiluted in antigen coated and blocked for 2 hours at room temperature. After washing binding of the monoclonal antibody was detected by incubation with a secondary antibody of goat anti-mouse IgG+IgM (H+L) conjugated to alkaline-phosphatase (Jackson, catalogue number 115-055-044), diluted 1/1000 in phosphate buffered saline. Wells were washed and positive binding was developed with p-nitrophenylphosphate substrate solution for 20-25min before stopping with 50 μl of 2M sodium hydroxide. Optical density absorbances were read at 405 nm.

### Results

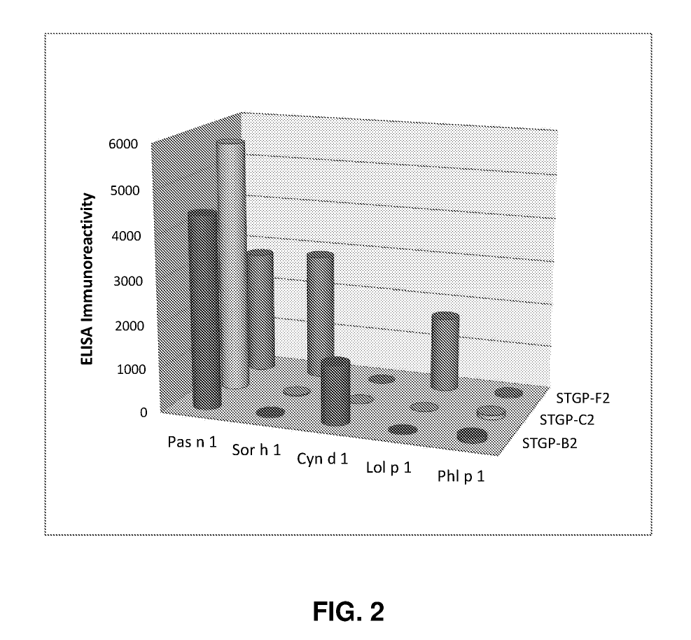

As can be observed in FIG. 2, mAb's from the STGP-B2 hybridoma bind both Pas n 1 and Cyn d 1 grass pollen allergens, with higher binding to Pas n 1. Conversely, mAb's from the STGP-F2 hybridoma show binding with Pas n 1 and Sor h 1 with similar levels of reactivity for each, as well as lower level binding with Lol p 1 grass pollen allergens. Monoclonal antibodies from the STGP-C2 hybridoma specifically bound Pas n 1 and not other group 1 pollen allergens tested. All of the three clones demonstrated little or no mAb binding reactivity with the Ph1 p 1 allergen from Timothy grass.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein are incorporated herein by reference.

### REFERENCES

Baldo B. A. (1983), Standardization of allergens, examination of existing procedures and the likely impact of existing procedures and the likely impact of new techniques on the quality control of extracts, Allergy, 38: 535-546.
Chase A. (1995), Revision of the Hitchcocks Manual of Grasses of the United States, US Dept Agriculture Miscel Publication 200, p 605.
Felsenstein, J. (1989), PHYLIP—Phylogeny Inference Package (Version 3.2). Cladistics 5: 164-166.
King T. P. (1976), Chemical and biological properties of some atopic allergens, Adv Immunol, 23: 77-105.
King and Norman (1962), Isolation studies of allergens from ragweed pollen, Biochemistry, 1: 709-720.
Matin B. G. et al. (1985), Cross-allergenicity among the grasses, Ann Allergy, 54: 99-104.
Petersen A. et al. (1993), Characterisation of grass Group II allergens in timothy grass pollen, J. Allergy Clin. Immunol., 92: 789-796.
Phillip J. W. et al. (1979), Bahia grass pollen—an important aeroallergen, J. Allergy Clin Immunol, 63: 192-193.
Sweeny M. et al. (1994), Immunodetection and comparison of Melaluca, Bottlebrush, and Bahia pollens, Int Arch Allergy Appl Immunol, 105: 289-296.
Thompson et al. (1994), CLUSTAL W: Improving the sensitivity of progressive multiple sequence alignment through sequence weighting, position-specific gap penalties and weight matrix choice, Nucleic Acids Research, 22: 4673-4680.
Tovey and Baldo (1987), Characterization of allergens by protein blotting, Electrophoresis, 8: 452-463.

TABLE 2
Primer sequences used for amplification and
cloning Pas n 1

| | |
|---|---|
| BG1for1 | Gggccoccgaaggtggcnccoggcaaraacatcac* |
| BG1for2 | gggcccccgaaggtggcncccggc |
| BG1rev1 | ggtgcctccctcggtagtgaggcggacgg |
| BG1rev2 | cccttcttggccatggcgccgaacgc |
| BG1rev4 | cggatcaagtgcatgcgcatgatgcatgg |
| BG1rev6 | gcttgcaagcttctagaactggacgttggaattg |

*degenerate nucleotides r = ag and n = agct

1. A method for detecting a Pas n 1 protein and/or measuring the level of the Pas n 1 protein in a sample, wherein the Pas n 1 protein comprises an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, said method comprising the step of contacting the sample with an antibody or antibody fragment that binds the Pas n 1 protein, fragment or derivative for a time and under conditions sufficient to allow the formation of a detectable antibody- or antibody fragment-protein complex.

2. The method of claim 1, wherein the sample comprises one or a plurality of other grass pollen-derived proteins in addition to said Pas n 1 protein.

3. The method of claim 2, wherein the one or plurality of other grass pollen-derived proteins are selected from the group consisting of a Sor h 1 protein, a Cyn d 1 protein, a Lol p 1 protein, a Lol p 5 protein, a Lol p 11 protein, a Phl p 1 protein, a Dac g 1 protein, an Ant o 1 protein, a Pha a 1 protein, a Poa p 1 protein, and a Pas n 13 protein.

4. The method of claim 3, wherein the one or plurality of other grass pollen-derived proteins includes a Sor h 1 protein and/or a Cyn d 1 protein.

5. The method of claim 2, wherein the antibody is capable of binding both the Pas n 1 protein and the one or a plurality of the other grass pollen-derived proteins.

6. The method of claim 5, wherein the antibody is capable of binding the Pas n 1 protein and either: (i) a Sor h 1 protein (or a fragment, variant or derivative thereof); (ii) a Cyn d 1 protein (or a fragment, variant or derivative thereof); or (iii) a Sor h 1 protein and a Cyn d 1 protein (or a fragment, variant or derivative thereof).

7. A method for detecting a Cyn d 1 protein and/or a Sor h 1 protein and/or measuring the level of the Cyn d 1 protein and/or the Sor h 1 protein in a sample, said method comprising the step of contacting the sample with an antibody or antibody fragment that binds and/or is raised against a Pas n 1 protein comprising an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, for a time and under conditions sufficient to allow the formation of a detectable antibody- or antibody fragment-protein complex.

8. The method of claim 7, wherein the sample comprises one or a plurality of other grass pollen-derived proteins in addition to said Cyn d 1 protein and/or said Sor h 1 protein.

9. The method of claim 8, wherein the one or plurality of other grass pollen-derived proteins are selected from the group consisting of a Lol p 1 protein, a Lol p 5 protein, a Lol p 11 protein, a Phl p 1 protein, a Dac g 1 protein, an Ant o 1 protein, a Pha a 1 protein, a Poa p 1 protein, a Pas n 1 protein and a Pas n 13 protein.

10. The method of claim 7, wherein the method is for determining a relative and/or absolute amount of each of the proteins in the sample.

11. The method of claim 7, wherein the sample is an environmental sample.

12. (canceled)

13. The method of claim 1, wherein the sample is, or is derived from, a pharmaceutical composition for immunotherapy.

14. The method of claim 1, wherein the sample is, or is derived from, a diagnostic composition.

15. The method of claim 13, wherein the method is performed to batch standardize the pharmaceutical composition or the diagnostic composition.

16. An antibody or antibody fragment that binds and/or is raised against a Pas n 1 protein comprising an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, wherein the antibody or antibody fragment also binds one or a plurality of other grass pollen proteins.

17. A method of producing an antibody or antibody fragment including the step of producing and/or selecting an antibody or antibody fragment that binds and/or is raised against a Pas n 1 protein that comprises an amino acid sequence as set forth in SEQ ID NO:2 or 4 or a fragment or derivative thereof, or an amino acid sequence having at least 86%, 87%, 88%, 89%, 90%, 91%, 92%, 93%, 94%, 95%, 96%, 97%, 98% or 99% or more sequence identity to SEQ ID NO:2 or 4 over the entire length of the SEQ ID NO:2 or 4 amino acid sequence, whereby the antibody or antibody fragment also binds one or a plurality of other grass pollen proteins.

18. An antibody or antibody fragment produced by the method of Claim 17.

19. The antibody or antibody fragment of claim 16, wherein the antibody or antibody fragment is a recombinant antibody or antibody fragment.

20. The antibody or antibody fragment of claim 19, wherein the recombinant antibody or antibody fragment comprises an amino acid sequence that is modified or engineered to facilitate binding to said one or more other grass pollen proteins.

21. The antibody or antibody fragment of claim 18, wherein the one or a plurality of other grass pollen proteins includes a Sor h 1 protein and/or a Cyn d 1 protein.

22. (canceled)

23. A composition or kit comprising the antibody or antibody fragment of claim 16.

24. The composition or kit of claim 23, further comprising one or more reagents that facilitate detection and/or measurement of a complex comprising the antibody or antibody fragment and the protein(s).

25. The method of claim 1, wherein the method is for determining a relative and/or absolute amount of each of the proteins in the sample.

26. The method of claim 1, wherein the sample is an environmental sample.

27. The method of claim 1, wherein the sample is, or is derived from, a pharmaceutical composition for immunotherapy.

28. The method of claim 1, wherein the sample is, or is derived from, a diagnostic composition.

29. The method of claim 27, wherein the method is performed to batch standardize the pharmaceutical composition.

30. The method of claim 28, wherein the method is performed to batch standardize the diagnostic composition.

31. The method of claim 17, wherein the one or a plurality of other grass pollen proteins includes a Sor h 1 protein and/or a Cyn d 1 protein.
